(19) United States
(12) Patent Application Publication
(10) Pub. No.: US 2025/0244430 A1
(43) Pub. Date: Jul. 31, 2025

(54) SYSTEMS AND METHODS FOR RF PULSE DESIGN FOR MRI PULSE SEQUENCES

(71) Applicant: Case Western Reserve University, Cleveland, OH (US)

(72) Inventors: William Grissom, Shaker Heights, OH (US); Mark Griswold, Shaker Heights, OH (US); Madison M. Albert, Shaker Heights, OH (US)

(21) Appl. No.: 19/035,435

(22) Filed: Jan. 23, 2025

Related U.S. Application Data
(60) Provisional application No. 63/624,890, filed on Jan. 25, 2024.

(51) Int. Cl. G01R 33/561; A61B 5/055; G01R 33/56

(52) U.S. Cl. CPC G01R 33/5617; A61B 5/055; G01R 33/5608

(57) ABSTRACT

Systems and methods for radiofrequency (RF) pulse design are provided. A method includes defining a target slice profile across a slice dimension and simulating a first echo having a first slice profile generated in response to a first one or more RF pulses. The method also includes simulating a second echo having a second slice profile generated in response to a second one or more RF pulses. The method further includes determining pulse parameters of at least one RF pulse for acquiring MRI data from the imaging target by reducing a cost function. The cost function includes a comparison term that calculates a difference between the first slice profile and the second slice profile and a target term that calculates a difference between the target slice profile and at least one of the first slice profile and the second slice profile.

### CROSS-REFERENCE TO RELATED APPLICATIONS

This application claims priority under 35 U.S.C. § 119 to U.S. Provisional Patent Application No. 63/624,890 filed on Jan. 25, 2024. The entire contents of which is incorporated herein by reference.

### STATEMENT REGARDING FEDERALLY SPONSORED RESEARCH

N/A

### BACKGROUND

MRI pulse sequences use shaped radiofrequency (RF) pulses to localize measured signals to desired slices (for two-dimensional or multi-slice two-dimensional scans) or slabs (for 3D scans) of tissue in the body. These pulses are conventionally designed using Fourier analysis, the Shinnar-Le Roux (SLR) algorithm, or the optimal control algorithm. For simplicity, these designs implicitly ignore the initial magnetization state or assume that magnetization is in an ideal uniform state prior to the pulses. For example, excitation pulse design assumes that magnetization starts out in a uniform equilibrium state. However, in practice the magnetization is rarely in a uniform state prior to application of an RF pulse. This leads to slice profiles that deviate from those desired, which can cause ghosting and blurring in applications like turbo spin echo (TSE). Moreover, RF pulse design that considers the temporal evolution of the signal typically only do so at the center of the slice. Thus, systems and methods for improving pulse design are desired.

### SUMMARY OF THE DISCLOSURE

The present disclosure addresses the aforementioned drawbacks by providing a system and method for RF pulse design for MRI pulse sequences.

In some aspects, a method for using a processor to prepare a magnetic resonance imaging (MRI) pulse sequence for performing imaging of a patient is presented. The method includes defining a target slice profile across a slice dimension of an imaging target. The method further includes simulating a pulse sequence having a plurality of RF pulses; simulating the pulse sequence includes calculating a plurality of slice profiles produced in response to the plurality of RF pulses. The method also includes determining pulse parameters of each of the plurality of RF pulses by reducing a cost function, which includes a comparison term that calculates differences between the plurality of slice profiles and a target term that calculates a difference between the target slice profile and at least one of the plurality of slice profiles.

In other aspects, a method for using a processor to prepare an MRI pulse sequence for performing imaging of a patient is provided. The method includes defining a target slice profile across a slice dimension of an imaging target. The method also includes simulating a first echo that has a first slice profile generated at a first echo time in response to a first one or more RF pulses. The method also includes simulating a second echo that has a second slice profile generated at a second echo time in response to a second one or more RF pulses. The method further includes determining pulse parameters of at least one of the first one or more RF pulses or the second one or more RF pulses for acquiring MRI data from the imaging target by reducing a cost function. The cost function includes a comparison term that calculates a difference between the first slice profile and the second slice profile as a function of the slice dimension and a target term that calculates a difference between the target slice profile and at least one of the first slice profile and the second slice profile as a function of the slice dimension.

In still other aspects, a computer-readable storage medium that has a stored computer program is provided. When the program is executed by a computer processor, it causes the processor to carry out steps that include receiving a target slice profile across a slice dimension of an imaging target. The steps also include simulating a first echo having a first slice profile generated at a first echo time in response to a first one or more RF pulses. The steps also include simulating a second echo having a second slice profile generated at a second echo time in response to a second one or more RF pulses. The steps also include determining pulse parameters of at least one of the first one or more RF pulses or the second one or more RF pulses for acquiring MRI data from the imaging target by reducing a cost function. The cost function includes a comparison term that calculates a difference between the first slice profile and the second slice profile as a function of the slice dimension. The cost function also includes a target term that calculates a difference between the target slice profile and at least one of the first slice profile and the second slice profile as a function of the slice dimension.

These are but a few, non-limiting examples of aspects of the present disclosures. Other features, aspects and implementation details will be described hereinafter.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 provides a flowchart setting forth steps of an example process that can be used to design radiofrequency (RF) pulses in accordance with the present disclosure.

FIG. 2 provides an example optimization process that can be used to simulate pulse sequences to design RF pulses.

FIG. 3 shows an example of three RF pulses, designed jointly in accordance with the present disclosure.

FIG. 4A provides example simulated data plotting magnetization profiles.

FIG. 4B provides example simulated data plotting magnetization profiles in comparison with those shown in FIG. 4A.

FIG. 5A provides example simulated RF pulses and magnetization profiles for a simulated turbo spin echo pulse sequence designed in accordance with the present disclosure.

FIG. 5B provides example simulated magnetization profiles for a simulated turbo spin echo pulse sequence designed in accordance with the present disclosure.

FIG. 6 provides example magnetization profiles measured in a phantom using a turbo spin echo pulse sequence designed in accordance with the present disclosure.

FIG. 7 provides example phantom images acquired using a turbo spin echo pulse sequence designed in accordance with the present disclosure.

FIG. 8 provides example in vivo images acquired using a turbo spin echo pulse sequence designed in accordance with the present disclosure.

FIG. 9 is a block diagram of an example magnetic resonance imaging (“MRI”) system that can implement the methods described in the present disclosure.

FIG. 10 is a block diagram of an example MRI system that can implement the methods of the present disclosure.

FIG. 11 is a block diagram of example components that can implement the system of FIG. 10.

### DETAILED DESCRIPTION

Before any aspects of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

In magnetic resonance imaging (MRI), radiofrequency (RF) pulses are typically designed to excite or otherwise localize signals with a desired slice profile. This slice profile often describes a magnetization profile described along a slice dimension (e.g., z), and is typically defined as a rectangular or nearly rectangular slice (e.g., as in 2D imaging) or slab (e.g., as in 3D imaging). Conventionally, pulse design is independently performed for each pulse of a pulse sequence. Moreover, typical pulse design fails to account for the spatially varying spin history imparted along the slice profile from previously played pulses. For example, the signal evolution is typically only considered at the center of the slice. This simplified pulse design process leads to slice profile differences across echoes (e.g., a series of echoes within an echo train or echoes from two different pulse sequences).

Most pulse design is currently performed to optimize a single RF pulse. In contrast, the present disclosure provides systems and methods that allow for joint pulse design. In some implementations, each of the individual pulses (e.g., for a whole pulse sequence) can be concatenated together to form a vector for use in the optimization. In contrast to previously described methods, the design is constrained (e.g., to achieve similar slice profiles) based on intermediate magnetization states that occur throughout the duration of the concatenated pulse. Thus, it is not necessary to assume that the magnetization is homogeneous at the time each pulse is performed.

Moreover, previously described approaches for joint design of multiple RF pulses are only applicable for RF pulses whose magnetization profiles are coupled together via incomplete relaxation. Such methods rely on sequence-specific recursive algebra that is specific to the design problem. However, by simulating the pulse sequence (e.g., Bloch simulations or extended phase graph calculations), the methods described herein provide a general process that can advantageously be applied to any pulse sequence. For example, the methods can be applied for turbo spin echo (TSE) sequences and other echo train sequences to reduce blurring and ghosting. As other examples, the joint pulse design can be applied where slice profile inconsistencies are known to contribute to image artifacts, such as for magnetic resonance fingerprinting (MRF), fluid-attenuated inversion recovery (FLAIR), and spin echo (SE) echo planar imaging (EPI).

The present disclosure provides systems and methods to jointly design a group of RF pulses while accounting for the z-dependent spin history in order to reduce slice profile differences across echoes. In this way, the separate RF pulses are jointly designed with respect to both temporal (spin history) and spatial (e.g., slice profile) magnetization profiles. The pulse design accounts for how the shapes of the slice profiles interact over time. In contrast, standard pulse design fails to account for the true spatially varying spin history imparted by previously performed pulses.

As one non-limiting example, the systems and methods described herein can be applied to provide two pulse sequences (e.g., a gradient recalled echo (GRE) and a spin echo (SE)) that achieve slice profiles with reduced differences. Jointly designing these two pulse sequences can provide a tool for comparing pulse sequences across a shared slice profile or provide images of varying contrast for a shared slice profile.

As another non-limiting example, the joint pulse optimization can be applied to steady state sequences. For example, in a spoiled GRE sequence the imaged magnetization at each repetition time (TR) is proportional to the longitudinal magnetization. The first TR's excitation pulse is applied to fully relaxed/equilibrium magnetization, while subsequent TRs are applied to longitudinal magnetization that has been attenuated in a non-uniform pattern through the slice. This means that the signal profile will be modified by the previous pulses in a manner that the pulse designer did not anticipate. In this scenario, it is desirable to design each RF pulse based not only on the initial magnetization profile but on the steady state profile. Moreover, it is further desirable to design a set of variable RF pulses that could maintain the same slice profile starting at the first TR until steady state is reached.

As another non-limiting example, the described systems and methods can be applied in the context of 2D or 3D turbo spin echo (TSE) pulse sequences (e.g., SPACE or CUBE). TSE pulse sequences generally use refocusing pulses throughout the echo train. These refocusing pulses can have a 180° flip angle or a sub−180° flip angle to reduce patient heating. In many implementations, the flip angle varies throughout the pulse sequence to maintain high echo signal amplitudes based on balancing longitudinal and transverse relaxation. Usually the same pulse shape is used for every refocusing pulse throughout the echo train while the amplitude of each pulse is modulated to achieve the desired flip angle in the center of the slice or slab. The use of the same pulse shape results in varying slice profiles through the echo train. These inconsistencies lead to unanticipated signal difference, which cause ghosting or aliasing and blurring in reconstructed images. The blurring caused by T₂ decay can be reduced prospectively by optimizing the flip angle throughout the train or corrected retrospectively by scaling the measured signal based on the estimated T₂ value and the echo's previous flip angle. However, these methods do not correct artifacts caused by slice profile inconsistencies and only work for one T₁/T₂ pair. Instead, the pulses of the echo train can be jointly optimized, as described herein, to provide more similar slice profiles, which may reduce blurring and ghosting artifacts in the combined data.

Thus, the present disclosure provides a method to jointly design all the individual RF pulses within the echo train. This joint pulse design can be based on a comprehensive model of the magnetization pathways evolving between echoes to enhance signal stability. In some implementations, the magnetization pathways can be modeled using extended phase graphs (EPG), which provides high accuracy. In other implementations spinor-EPG provides a differentiable and computationally convenient forward model of the magnetization. In other implementations, Bloch simulations can be used to model the magnetization pathways.

In some implementations, the pulse design described herein accounts for soft or time-varying pulses in the spinor domain to calculate the magnetization profile across spatial locations at each echo. The model can be designed to be fully differentiable, which allows for convenient joint optimization of pulse shapes that promotes slice profile similarity. These consistent slice profiles can be used to decrease blurring and ghosting (e.g., in TSE sequences) or otherwise match slice profiles between sequences or throughout a pulse sequence.

The evolution of the magnetization can be calculated as the RF pulses are simulated, accounting for off-resonance and relaxation effects that are experienced by the spins. The simulated signal or slice profiles occurring at each echo (e.g., for each excitation, TR, or TE) can be normalized by an expected signal amplitude for an idealized sequence and compared to one another so that they can be made maximally identical via the pulse design. The design may further incorporate pulse-specific target rotation profiles such as a beta profile for a refocusing pulse, to stabilize the design.

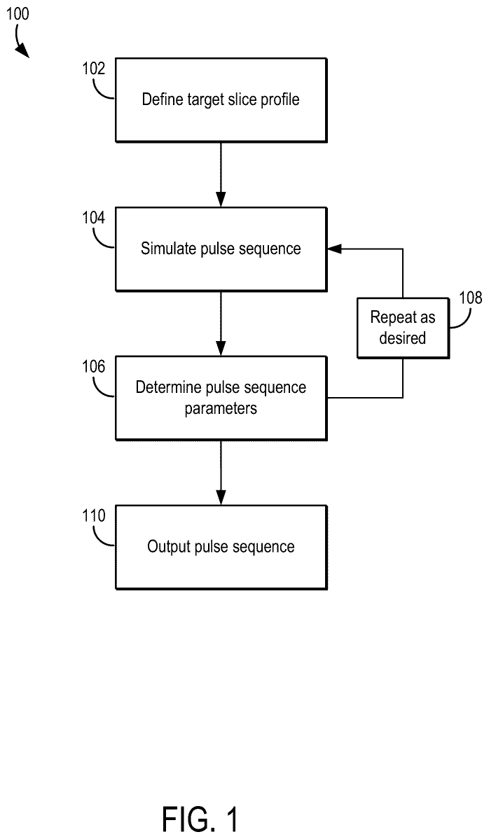

FIG. 1 provides a flowchart setting forth steps of a process 100 that can be used for RF pulse design to prepare an MRI pulse sequence. The process 100 can be used to design a selective RF pulse that produces a desired magnetization profile that has been influenced by the RF pulse and one or more previous selective RF pulses. As a non-limiting example, process 100 can be used to design a refocusing pulse of a SE pulse sequence in which the magnetization profile that occurs at the echo time has been influenced by the refocusing pulse and a temporally preceding excitation pulse. Process 100 can also be used to design several selective RF pulses that produce desired magnetization profiles, each influenced by one or more preceding RF pulses. As a non-limiting example, process 100 can be used to jointly design RF pulses of a TSE echo train where the magnetization profile at each echo is influence by all of the preceding pulses.

In general, process 100 performs a joint RF pulse design to improve similarity between two or more slice profiles. These slice profiles describe a complex- or real-valued magnetization as a function of space in the slice direction (e.g., z). Thus, slice profiles may also be referred to as signal profiles or magnetization profiles. The two or more slice profiles may occur sequentially in time (e.g., at different echo or repetition times) within the same pulse sequence or may occur in separate pulse sequences.

An optimization is described that updates the pulse parameters (e.g., B₁ waveform) of two or more spatially selective RF pulses based on simulated slice profiles associated with the RF pulses. A cost function can be defined that promotes similarity between the slice profiles. The system model can be described differentiably to facilitate convenient and computationally inexpensive optimization.

Process 100 can also include normalization or scaling of each magnetization profile to have similar amplitudes such that the shapes of the slice profiles can be directly compared within the optimization pipeline.

Process 100 can advantageously be applied for any desired pulse sequence to allow for a variety of applications. As a non-limiting example, the joint RF pulse design can be performed for a combination of excitation and refocusing pulses, which may include variable flip angles, in a 2D or 3D fast spin echo (FSE) or turbo spin echo (TSE) sequence. As another example, the pulse sequences can include two or more separate pulse sequences, such as a GRE pulse sequence and a SE pulse sequence, such that the RF pulses are designed jointly to achieve similar target profiles at each respective echo time. As another example, process 100 can include designing a steady state or transient-to-steady-state signal profiles in a balanced or unbalance or spoiled GRE sequence to achieve similar target profiles for each echo throughout the pulse sequence. As yet another example, the pulse sequence can be designed to produce a stimulated echo sequence. As another non-limiting example, the RF pulse design can be applied to generate a magnetization preparation stage of a pulse sequence, which can be jointly designed with subsequence pulses that provide the imaging portion of the pulse sequence. For example, the RF pulses can be designed to invert magnetization in a slice for T₁ weighting or fat suppression (as in an inversion recovery pulse), and then the imaging pulses can be designed to have the same profile as experienced by the magnetization preparation pulses.

In process block 102, a target slice profile can be defined. This target slice profile can describe the desired magnetization profile M (z), as a function of the slice dimension. In some implementations, the target slice profile can be described by a shape and amplitude of the transverse magnetization (M_{xy}(z)). The amplitude of the transverse magnetization may be defined in terms of a flip angle (e.g., 90°). In some implementations the target slice profile can be further described by the longitudinal magnetization (M_{z}(z)). Practically, M can be treated as a vector, where each entry represents a discretized positions in z.

In some implementations, the target slice profile can be described with respect to the amplitude (e.g., |M_{xy}(z)|) such that the phase is ignored. For example, the target slice profile may be defined to have a constant magnitude for a rectangular slice shape. In other implementations, the target slice profile can be additionally described by a phase (e.g., ∠M_{xy}(z) or Mₓ(z) and M_{y}(z)). For example, the target phase may be defined as a constant or minimally changing phase across the rectangular slice profile. As another example, the target slice profile can be defined to have a non-linear or other phase pattern in order to reduce peak RF power of the associated RF pulses. As another example, the target slice profile can be defined to have a non-constant amplitude or phase with a spatial function that enables sub-slice spatial encoding.

In some implementations, the target slice profile can be defined for a desired echo. For example, the target profile can be defined with respect to the refocusing pulse of a spin echo sequence. As another example, the target slice profile can be defined with respect to one of the echoes of an echo train (e.g., the first echo or last echo). As another example, the target profile can be defined with respect to the imaging echoes of a magnetization prepared sequence.

The target slice profile can be represented in several different ways. In general, the target slice profile can describe a target rotation as a function of another parameter of the system (e.g., position in z, resonance frequency offset, or transmit RF field amplitude). For example, the target slice profile can be defined for each associated RF pulse in terms of an alpha profile, a beta profile, or a combination thereof. These parameters describe the rotation operation applied by an RF pulse, independent of the initial state of the magnetization when the RF pulse is applied. These profiles may be associated with spinor parameter profiles that can be used to mathematically describe the associated RF pulses, as will be described further below. The use of the spinor profiles provides complex-valued parameters (e.g., α and β) that compactly describe the rotation applied by the RF pulse and gradient pulse. In some implementations, the rotation can alternatively be described using a 3×3 rotation matrix. In other implementations, the target slice profile can be described as a desired flip angle pattern.

In process block 104, the pulse sequence or multiple pulse sequences are simulated to calculate slice profiles that are produced in response to the RF pulses in the pulse sequence. The magnetization or slice profiles can be simulated for desired echoes of the pulse sequence (e.g., for various excitations, TRs, or TEs) or at the respective echo times within separate pulse sequences. This simulation can be performed in order to characterize the evolution of the magnetization that results from relaxation effects and the RF pulses, including the most recent RF pulse and other preceding RF pulses. In this way, simulating each slice profile can account for the magnetization profile that occurs at the time of the associated RF pulse, including effects of previously experienced slice profiles. Thus, the simulation can be performed to account for the full spin history of each pulse sequence. In some implementations, the simulation can further account for other system deviations, such as off resonance or B₁ inhomogeneity.

A desired pulse sequence can be defined prior to simulation. For example, the desired pulse sequence can include a number and type of RF pulses (e.g., echo train length), and timing parameters of the sequence (e.g., TEs, TR, inversion time). Each RF pulse can be represented by a finite number of points in time (e.g., 128 points) that can later be interpolated. The pulse sequence can be simulated through time for the full pulse sequence or a portion of interest of the pulse sequence.

Simulating the pulse sequence may also include defining a reference subject or imaging target. Defining this reference can include defining associated relaxation parameters (e.g., T₁ and T₂). As a non-limiting example, the reference can be defined as a homogeneous sample with tissue-like (e.g., water, white matter, gray matter, adipose tissue, muscle) relaxation parameters. As another non-limiting example, the reference can be defined by measuring or accessing a reference tissue or anatomical model (e.g., a database of brain images). As another example, the reference can be defined by acquiring phantom images. As another example, the reference can be defined for a specific subject by measuring tissue parameters for the subject, in which the optimization may be performed in real time or prospectively performed for the subject.

In general, simulating the pulse sequence can be performed to calculate the magnetization realized in response to RF pulses. Several different methods can be used to simulate the pulse sequence to calculate the resulting slice profiles. As one non-limiting example, a Bloch simulation can be used. As another example, extended phase graph (EPG) methods can be used. For example, EPG can be used to produce a forward model of the system, which relates the RF pulses with the slice profile using differentiable tensor operations. In some implementations, the RF pulses of the pulse sequence can be described by spinor profile parameters as in the Shinnar-Le Roux algorithm. An example implementation will be further described below.

As one non-limiting example, simulating the pulse sequence includes generating a forward model of the system based on the EPG method. The differentiable EPG model takes the RF pulses (e.g., an excitation RF pulse and a string of refocusing pulses) as input. The echo spacing time, the relaxation constants T₁ and T₂, and the spatial locations at which to calculate the magnetization are also input to the model. The forward model returns the complex magnetization profile at each echo in the sequence across the provided spatial locations.

In some implementations, the RF rotations can be applied using a 3×3 algorithm, as is done in the standard EPG algorithm. In other implementations, the spinor-EPG algorithm can be used to apply rotations in SU (2), which provides a convenient representation in which the rotations are described using only two complex numbers, α and β, where |α|²+|β|²=1. Additionally, the spinor representation of an RF pulse can be combined with the hard or constant pulse approximation to easily calculate the spinor profiles from a slice selective time-varying pulse, as in the Shinnar-Le Roux (SLR) algorithm. In some implementations, the rotation from each RF pulse is assumed to occur instantaneously with no relaxation effects, and the slice select gradient and RF rotations are applied sequentially.

To model the evolving magnetization across spatial locations throughout the pulse sequence, a partitioned EPG can be performed, where the EPG calculation is performed in parallel for each spatial location. First, the excitation RF pulse can be applied to the extended phase graph state matrix using its spinor profile representation. Then, each part of the pulse sequence can be simulated sequentially for each echo. As a non-limiting example, for each echo, the sequence parts may include the following sequence: relaxation, a crusher gradient, a refocusing RF pulse (e.g., using its spinor profile representation), a crusher gradient, relaxation, and an echo. Then the F₀ state, which represents the measured M_{xy} signal, can be recorded at each echo.

In some implementations, the pulse sequence simulation can further include effects from system imperfections (e.g., B₀ field inhomogeneity, RF transmit magnetic field (B₁) amplitudes). These effects can be estimated or measured for a reference or specific subject and incorporated into the model. Thus, the optimization may be performed in real time to simulate the pulses for scan-specific parameters (e.g., a measured or estimated B₀ or B₁ map).

In some implementations, performing the pulse design in real time can be achieved using a machine learning algorithm. For example, process 100 can be repeated for many B₁ maps to train a machine learning algorithm (e.g., convolutional neural network, unrolled neural network, etc.) to perform the optimization in real time using a measured B₁ map. In other implementations, performing the pulse design in real time can employ analytical derivations to reduce the computation time required.

In process block 106, the slice profiles, which were calculated for the simulated pulse sequence in process block 104, can be used to determine the RF pulse parameters. These RF pulses can be jointly optimized to achieve a target slice profile, as defined in process block 102, at one or more of the echoes and promote similarity of the simulated slice profiles at each echo.

In some implementations, the joint RF pulse design can be performed by an optimization in which a cost function is defined. In general, the cost function includes a comparison term that calculates the difference between slice profiles and a target term that calculates the difference between one of the slice profiles and the target slice profile. To directly compare the slice profile shapes, the simulated slice profiles can be normalized or scaled based on their expected signal amplitudes. This scaling can be determined base, for example, on relaxation rates, flip angles, or other parameters that affect the signal amplitude (e.g., B₀ homogeneity and B₁ profiles). As non-limiting examples, the scaling can be determined using an EPG method or Bloch simulation.

The target term calculates a difference between the target slice profile and at least one of the simulated slice profiles. As a non-limiting example, the target term may be described by the form ∥M_{T}−D∥, where ∥·∥ represents a vector norm (e.g., L1 or L2 norm) used to calculate a loss or error (e.g., an L1 or L2 loss); M_{T} represents the magnetization profile at a selected echo, T; and D represents the target slice profile. In some implementations, M and D can be described by their transverse and longitudinal components. Thus, the target term may be described by the form ∥M_{T,xy}−D_{xy}∥+∥M_{T,z}−D_{z}∥. Moreover, the target term can be weighted or normalized. This weight can be used to directly compare the shape of the slice profile and can incorporate signal decay (e.g., relaxation) and flip angle. As a non-limiting example, the weight can be applied using a weighting matrix, W, that provides a pass band weight, stop band weight, and transition band wight. For example, W can be set to have a pass band weight equal to 100, stop band weight equal to 1, and transition band weight equal to 0. Thus, the target term may be written as ∥M_{T,xy}−D_{xy}∥_{W}+∥M_{T,z}−D_{z}∥_{W}.

The comparison term calculates differences between the slice profiles of interest to provide a regularization that promotes similarity between slice profiles. As a non-limiting example, the comparison term can be described by the form ∥Σₙ₌₁^{N}(Mᵣ−Mₙ)∥ where N represents the number of echoes to compare (e.g., the echo train length, N_{ETL}); Mᵣ represents the magnetization profile at a reference echo, r (e.g., the first echo); and Mₙ represents the magnetization profile at each echo, n.

In some implementations, each echo of the comparison term can be normalized or weighted according to the signal amplitude. In this way, the slice profile shapes can be compared directly. Thus, the comparison term can be written as ∥Σₙ₌₁^{N}Cₙ(Mᵣ−Mₙ)∥, where Cₙ represents a scaling factor for the n^{th} echo. For example, Cₙ may be determined based on relaxation and flip angle.

The target term and comparison term in the loss function can be weighted to tune the tradeoff between the two terms. Thus, the loss function can be written as:

$$ℒ = {‖ {M}_{T , xy} - {D}_{xy} ‖}_{W} + {‖ {M}_{T , z} - {D}_{z} ‖}_{W} + λ ‖ {\sum }_{n = 1}^{N} {C}_{n} ( {M}_{r} - {M}_{n} ) ‖$$

where λ controls the tradeoff between the target term and comparison term.

In some implementations, the comparison term can be implemented by measuring the rank of the combined magnetization profiles (e.g., a matrix combining the slice profiles) to promote similarly between profiles. The use of a rank-based approach (e.g., a singular value decomposition (SVD)) can advantageously simplify the computation or obviate the need to specify target patterns or characteristics for all the slice profiles. SVD is one method that can conveniently be used as a convex and differentiable way to measure the rank of a matrix. However, other methods can also be used.

As a non-limiting example, the comparison term can be implemented by forming a matrix of the simulated transverse magnetization profiles and promoting the matrix to be low rank (e.g., rank−1). When a rank−1 matrix is achieved, it corresponds to the vector outer product of a single slice profile that is scaled to match the other slice profiles. In some implementations, the optimization can be performed to reduce the rank by minimizing the sum of the singular values for n>1. In other implementations, the rank can be reduced using an indicator function that is equal to 1 for any non-zero singular value.

As a non-limiting example, the comparison term can be calculated as: λΣₙ₌₁^{N}σₙₙ(M_{xy}), where σ is a matrix of singular values of M_{xy}. This regularization term serves to promote similarity between signal profiles M_{xy} by encouraging them to have the same shape, while permitting different scalings.

The optimization performed in process block 106 to determine the pulse sequence parameters can be achieved with any appropriate optimization method. For example, a gradient-based algorithm can be used to iteratively reduce the cost function. In some implementations, the optimization can be performed using autodifferentiation, which may advantageously reduce the complexity of the computer program that performs the optimization by circumventing the need to explicitly determine derivatives. In other implementations, analytical derivatives (e.g., based on the chain rule) can be used to reduce the computational time and memory requirements of the optimization problem. In other implementations, a combination of autodifferentiation and analytical derivatives can be used. In still other implementations, a neural network can be trained to reduce the cost function (e.g., using an unrolled neural network).

The optimization may be performed iteratively. Thus, the process can be repeated in process block 108. The iterative optimization may be performed for a preset number of iterations or until a stopping criterion is met (e.g., the loss is below a given threshold).

The jointly designed RF pulses can be output in block 110 to provide a pulse sequence for use. The RF pulse parameters provide a description of the associated RF pulse. For example, the pulse parameters may include a pulse shape (e.g., a B₁ amplitude through time). In other implementations, the RF pulse parameters can provide a parameterization of the RF pulse, such as using flip angles, Fourier series, b-splines, wavelets, and so forth. These RF pulses can be performed in time to generate echoes at desired times (e.g., echo times). Thus, process block 110 can include providing a full pulse sequence. Process block 110 can also include performing the pulse sequence using an MRI system to acquire MRI data from an imaging target or target volume.

In some implementations, the pulse sequence may provide an echo train that samples k-space using a train of echoes. Thus, process block 110 may include stitching the data together to generate k-space data. The data from each echo may be scaled to account for T₂ decay or other amplitude effects to provide an image with reduced blurring or ghosting.

### EXAMPLES

### Example 1

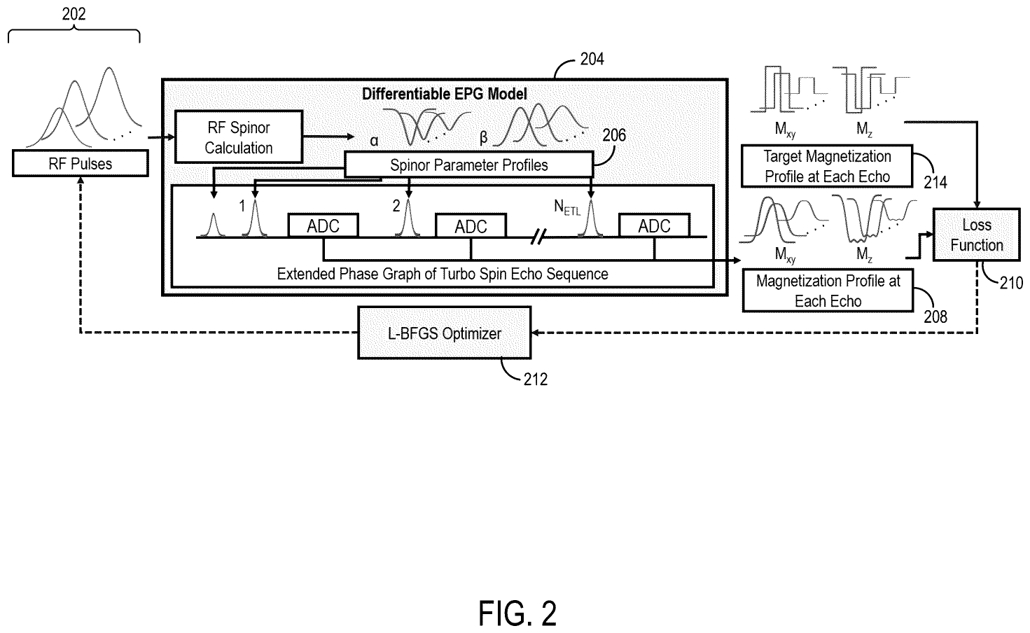

FIG. 2 provides an example implementation for simulating and optimizing pulse sequences with similar slice profiles. Thus, FIG. 2 may be used to perform process blocks 104 and 106, for example. In this example, a forward model is applied to design RF pulses 202 across echoes in a TSE sequence. However, the process can similarly be applied for other sequence types. The optimization includes a differentiable EPG model 204. The model uses the spinor representation 206 of the RF pulses from the SLR algorithm to apply RF rotations, enabling accurate, computationally efficient calculations of the slice profile at each echo 208. The spinor profiles can be calculated individually for each pulse across all the spatial locations. Then, EPG is performed at each spatial location looping through each echo (for N_{ETL} echoes) to simulate the magnetization at each spatial location for all the echoes. The loss function 210 is reduced or minimized using an L-BFGS optimizer 212 (e.g., in PyTorch) to design the excitation and refocusing RF pulses that match a target magnetization profile 214 and provide similar slice profile shapes throughout the echo train.

### Example 2

A short-TR (300 ms) single-echo SE pulse sequence and a GRE pulse sequence were jointly designed to achieve matching normalized signal profiles at each of the sequences' respective echo times

This example was motivated by the desire to eliminate slice profile differences as a confound when comparing spin-echo and gradient-echo BOLD fMRI acquisitions. In the pulse design, each pulse sequence was simulated for 25 TRs, assuming a T₁ of 1750 ms. The optimization was performed to increase similarity off the signal profiles at the TE of the 25th TRs to match between the sequences, after normalizing the profiles by the sequence's respective expected signal amplitude at the nominal flip angles. The design further incorporated a target spinor parameter (beta) slice profile for the refocusing pulse. The design loss terms were defined using 12-errors and were summed together with a variable weighting on the signal similarity terms.

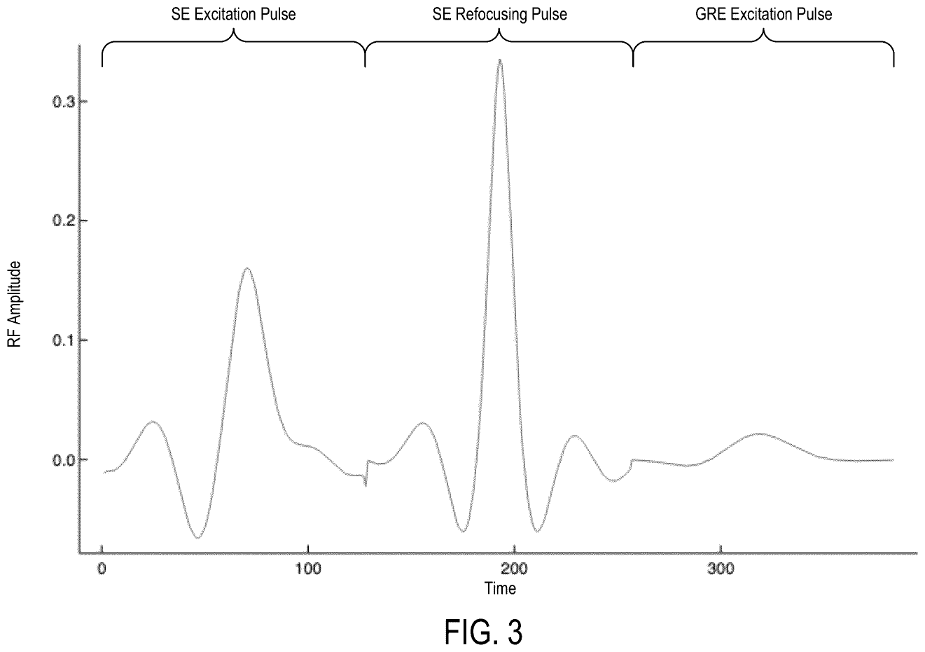

FIG. 3 shows the resulting waveforms jointly designed in accordance with the present disclosure. In FIG. 3, the three jointly designed RF pulses are displayed concatenated. The first pulse is the short-TR SE sequence's 147° excitation pulse; the second pulse is the SE sequence's 180° refocusing pulse; and the third pulse is the GRE sequence's 33° excitation pulse.

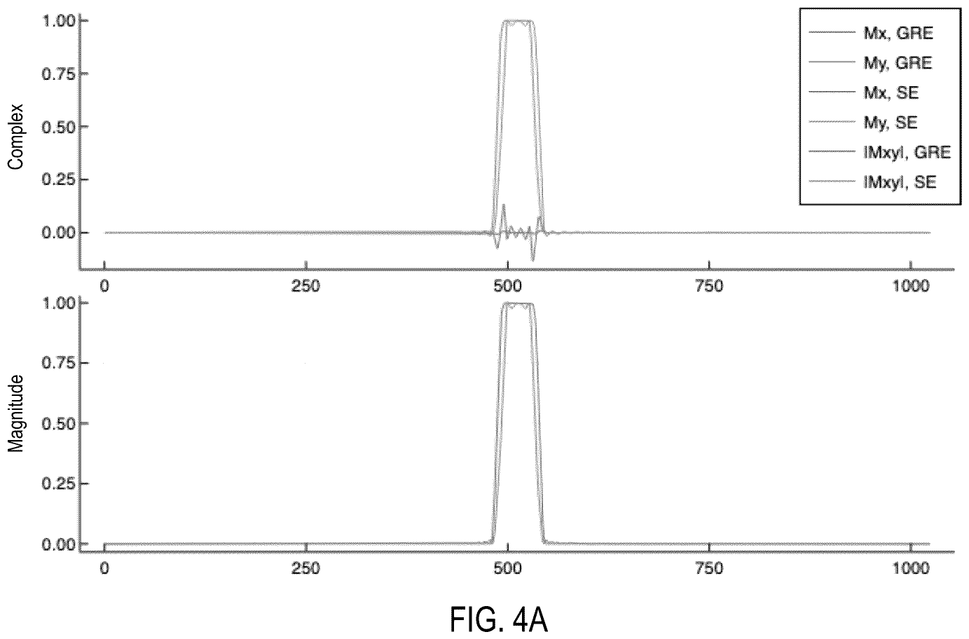

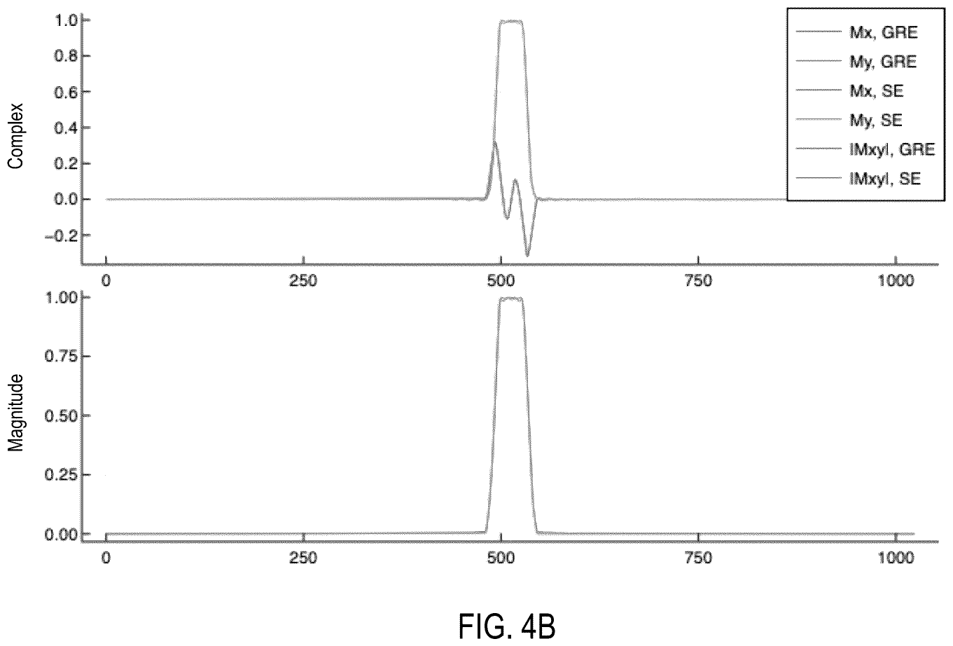

FIG. 4A shows the complex-valued (top) and magnitude (bottom) magnetization profiles (Mₓ and M_{y}) that occur at the echo times of the GRE and SE pulse sequences when the pulses are designed using standard methods that do not provide joint design. In this scenario, despite setting the target patterns to have the same nominal slice width, the lack of coupling between the designs leads to large differences in residual phase and width in the transition band.

FIG. 4B shows the complex-valued (top) and magnitude (bottom) magnetization profiles (Mₓ and M_{y}) that occur at the echo times of the GRE and SE pulse sequences when the pulses are jointly designed as described herein. The consistency of the magnetization profiles is greatly improved using joint RF pulse design.

### Example 3

The differentiable EPG-based forward model was implemented using PyTorch, and each step used differentiable tensor operations, which enabled the optimization the inputs to the model based on a loss function involving its outputs. In this example, the goal was to optimize each individual RF pulse for a TSE sequence. Each RF pulse was modeled as 128 points long, which was later interpolated. Each point in every pulse is allowed to take on any real value during the optimization.

The loss function was chosen to promote slice profile similarity at each echo while maintaining the signal magnitudes achieved at the center of the slice by a traditional TSE sequence. The loss was calculated on the final complex transverse magnetization slice profiles as:

$$ℒ = {‖ {M}_{xy} - {D}_{xy} ‖}_{W}^{2} + {‖ {M}_{z} - {D}_{z} ‖}_{W}^{2} + λ {\sum }_{n = 2}^{{N}_{ETL}} {σ}_{nn} ( {M}_{xy} )$$

where M is the simulated magnetization profile at each echo, and D is the target magnetization profile at each echo. The width of the target profile is defined as in the SLR algorithm based on the desired time-bandwidth of the pulse. The magnitude of the target pass band is the signal magnitude calculated in the center of the slice via EPG assuming a hard pulse with the desired flip angle train. ∥·∥² is the L2 norm. W is the weighting matrix for the normalization, with a pass band weight equal to 100, stop band weight equal to 1, and transition band weight equal to 0. σ is the matrix of singular values of M×y, and λ is a weighting factor.

The first two terms in this loss function ensure that the magnetization profiles maintain the desired bandwidth and magnitude. Minimizing the final term in the loss function maximizes the similarity between the magnetization profiles at each echo by penalizing all but the largest or first (n=1) singular values of the matrix of complex transverse magnetization profiles. If the magnetization profiles were all scaled versions of each other, there would only be one nonzero singular value. Thus, by minimizing all other singular values, the components of the magnetization profiles that are not similar are reduced.

The L-BFGS optimization algorithm to minimize the loss function was applied by changing the RF pulse shapes. The learning rate, number of iterations, and λ weighting factor were manually tuned and set to λ=0.2, learning rate=0.1, and 15 iterations.

The optimized RF pulses were compared to time-bandwidth matched RF pulses designed with the SLR algorithm. Both sequences were implemented in Pulseq with N_{ETL}=15, T_{ESP}=11.1 ms, TR=6 s, and TE=99.9 ms. The optimization was performed using relaxation values (T₁ and T₂) representative of white matter and grey matter to ensure signal profile similarity across a range of physiological relaxation values.

The target flip angle was set to 165° for the first refocusing pulse and 150° for the remaining refocusing pulses, which uses a known flip angle scheme to enhance signal stability. Target profiles were defined using a time-bandwidth product of 2.

In simulation, we calculated the magnitude of the integral of the scaled complex magnetization profile at each echo for each set of pulses for both white matter and grey matter. We scaled each profile by dividing by its maximum value to account for T₂ decay. Slice profile similarity was measured by calculating the standard deviation of the integral across echoes. We also visually compared the scaled |M_{xy}|, Mₓ, and M_{y} profiles across echoes to assess similarity. Additionally, we compared the decay of the maximum signal across echoes to ensure the optimized pulses produced a similar T₂ weighting.

All imaging experiments were performed at 3 T using a 20-channel phased array receive coil with IRB approval and volunteer consent. The slice profiles were measured in a homogeneous spherical phantom with 256 readout points, resolution=0.5 mm, and slice thickness=16 mm. Phantom and head 2D TSE images were acquired with a 22×22 cm2 field of view, matrix size of 512, and 4 mm slice thickness. Images were reconstructed using an inverse fast Fourier transform operation followed by a sum-of-squares coil combination. Magnitude slice profiles were reconstructed using a 1D inverse fast Fourier transform with sum-of-squares coil combination.

While T₂ decay correction was not applied, and the decay of the maximum magnitude at each echo was compared across pulses to ensure similar T₂ weighting in the imaging sequences. The full width at half maximum (FWHM) of the slice profile at each echo was calculated, and similarity was quantified as the standard deviation of the FWHM between echoes. Additionally, the slice profiles scaled by their maximum magnitude across echoes were visually compared to assess similarity.

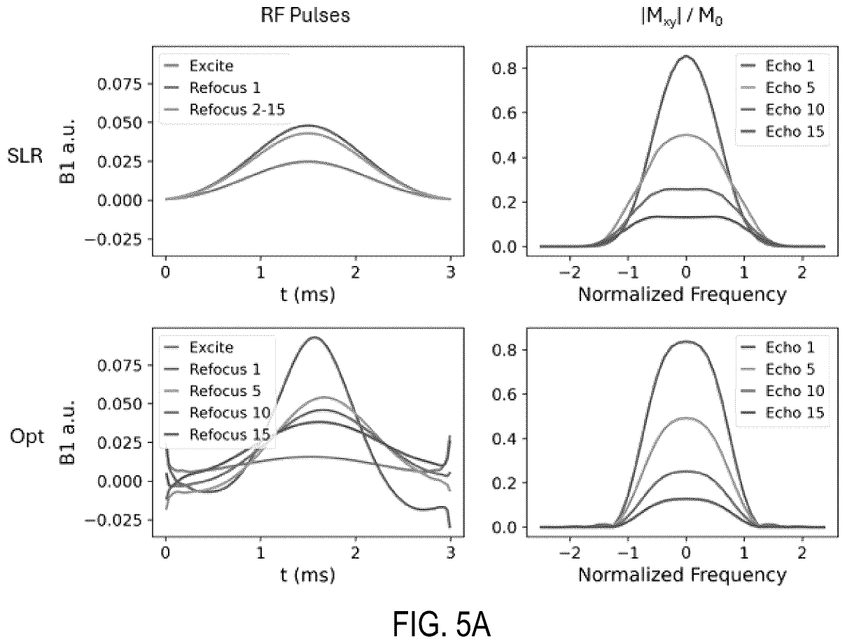

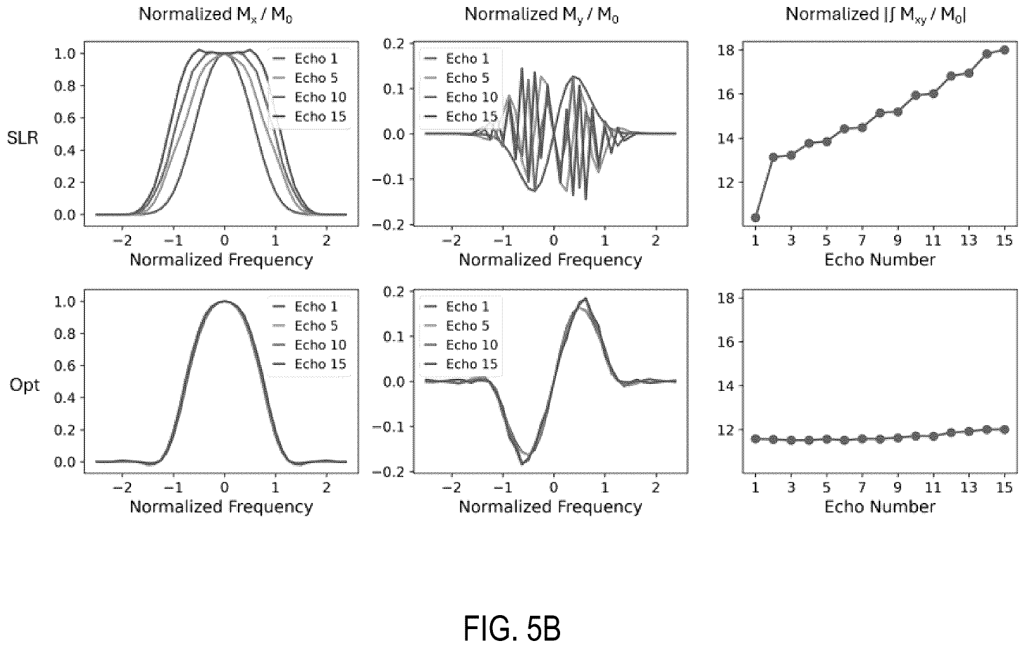

FIGS. 5A and 5B show the excitation and refocusing pulses (with time-bandwidth=2 and duration=3 ms) for the optimized (bottom) design compared to the SLR (top) design, in simulation. Magnetization profiles are plotted for white matter relaxation values. Four representative refocusing pulses and subsequent echo magnetization profiles from the echo train are shown. The RF pulses and |M_{xy}|/M₀ are shown in FIG. 5A, while the normalized Mₓ/M₀, normalized M_{y}/M₀, and normalized |∫M_{xy}/M₀ are shown in FIG. 5B. Optimized pulses showed 91% reduction in slice profile inconsistency compared to SLR pulses, as measured by the standard deviation of integrated complex magnetization normalized to center echo magnitude. Additionally, the similarity of the M_{y} profiles improved with the optimized pulses. Although M_{y} integrates to zero in simulation, the similarity minimizes partial volume effects.

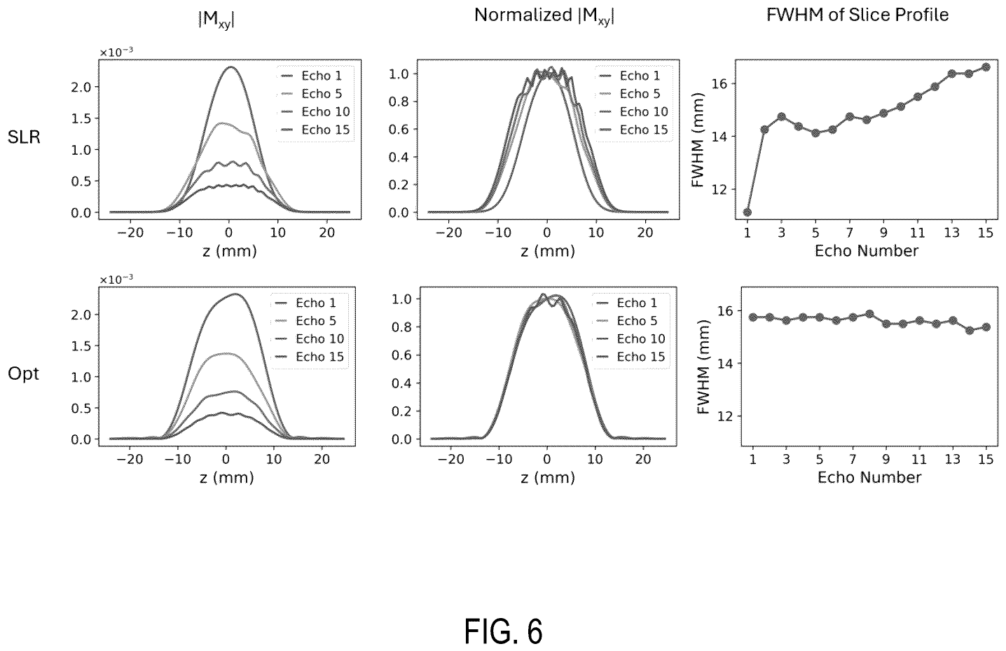

Slice profiles measured in the homogenous phantom confirmed the simulation results, with a 92% reduction in the FWHM standard deviation across echoes, as shown in FIG. 6. The slice profiles have thickness of 16 mm. Results are shown for SLR RF pulses (top) and optimized RF pulses (bottom). Four representative echoes from the echo train are shown (left). To visually compare similarity, each echo was also scaled based on its center value (middle). The standard deviation of the FWHM of the magnetization profile at each echo decreased by 92% with the optimized RF pulses (right).

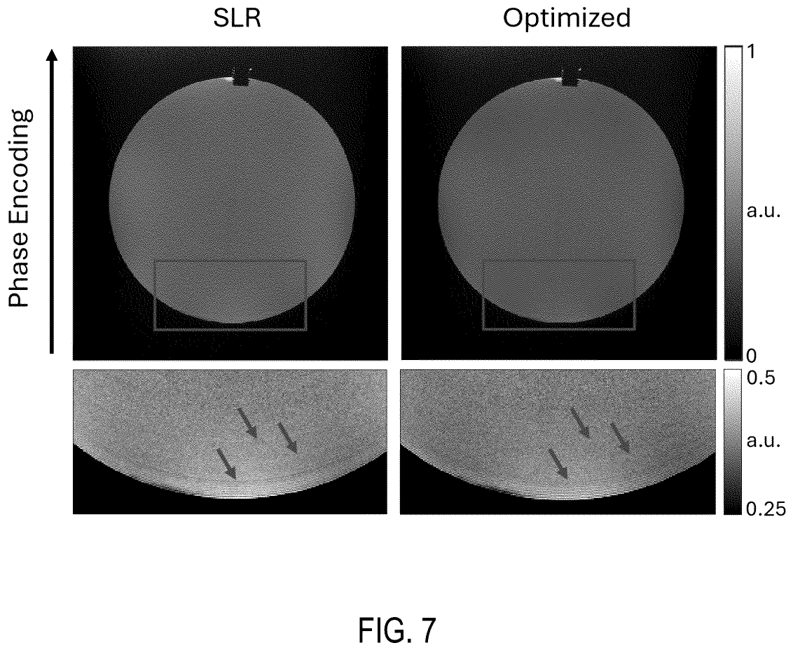

FIG. 7 compares bias-corrected images of a homogeneous phantom acquired with SLR RF pulses (left) and optimized RF pulses (right). Images acquired with the SLR pulses have at least three distinctly visible ghosting bands (arrows) in the phase encode (vertical) direction spaced N_{ETL}=15 voxels apart. The optimized RF pulses substantially reduced these ghosting bands, as indicated by the arrows. As expected, high-frequency Gibbs ringing is present in both images near the bottom edge of the phantom.

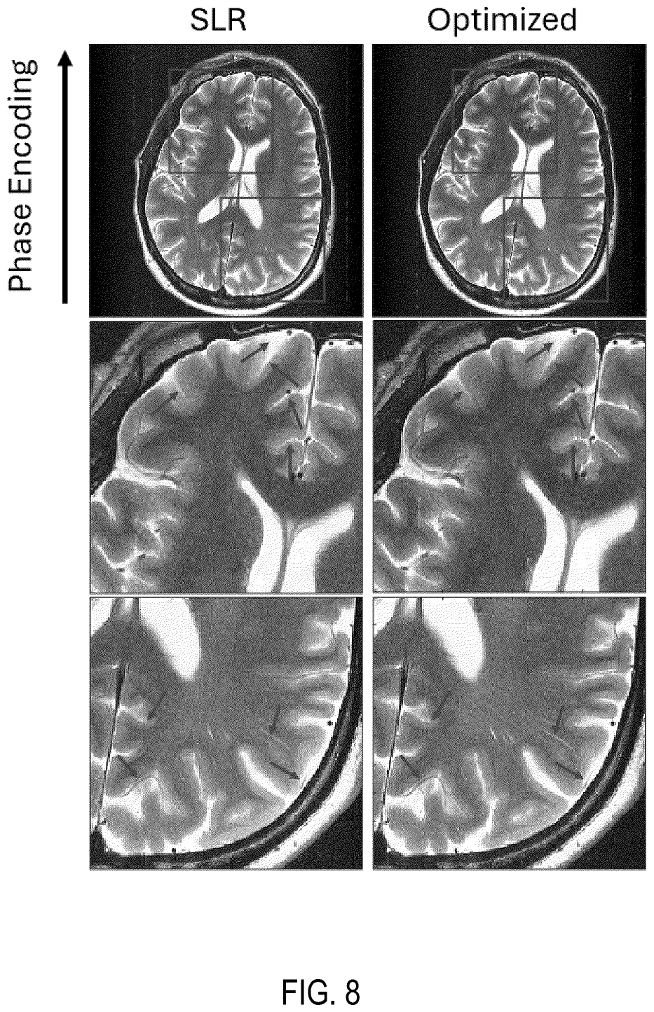

FIG. 8 shows T₂-weighted TSE head images acquired with both SLR RF pulses (left) and optimized RF pulses (right), in accordance with the present disclosure. The arrows indicate regions where the optimized pulses increased sharpness, resulting from improved signal stability with the optimized RF pulses. Increased sharpness is especially evident at edges between the cortex and CSF as well as veins, particularly those perpendicular to the phase-encoding direction. Similar results were achieved using optimized time-bandwidth 4 pulses.

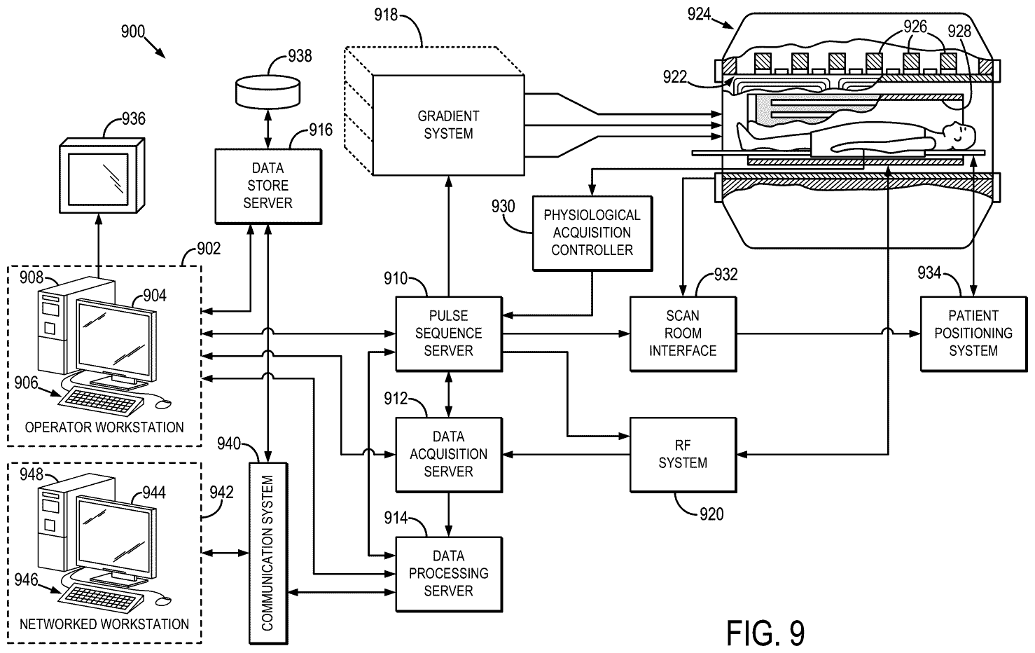

Referring particularly now to FIG. 9, an example of an MRI system 900 that can implement the methods described herein is illustrated. The MRI system 900 includes an operator workstation 902 that may include a display 904, one or more input devices 906 (e.g., a keyboard, a mouse), and a processor 908. The processor 908 may include a commercially available programmable machine running a commercially available operating system. The operator workstation 902 provides an operator interface that facilitates entering scan parameters into the MRI system 900. The operator workstation 902 may be coupled to different servers, including, for example, a pulse sequence server 910, a data acquisition server 912, a data processing server 914, and a data store server 916. The operator workstation 902 and the servers 910, 912, 914, and 916 may be connected via a communication system 940, which may include wired or wireless network connections.

The MRI system 900 also includes a magnet assembly 924 that includes a polarizing magnet 926, which may be a low-field magnet. The MRI system 900 may optionally include a whole-body RF coil 928 and a gradient system 918 that controls a gradient coil assembly 922.

The pulse sequence server 910 functions in response to instructions provided by the operator workstation 902 to operate a gradient system 918 and a radiofrequency (“RF”) system 920. Gradient waveforms for performing a prescribed scan are produced and applied to the gradient system 918, which then excited gradient coils in an assembly 922 to produce the magnetic field gradients (e.g., Gₓ, G_{y}, and G_{z}) that can be used for spatially encoding magnetic resonance signals. The gradient coil assembly 922 forms part of a magnet assembly 924 that includes a polarizing magnet 926 and a whole-body RF coil 928.

RF waveforms are applied by the RF system 920 to the RF coil 928, or a separate local coil to perform the prescribed magnetic resonance pulse sequence. Responsive magnetic resonance signals detected by the RF coil 928, or a separate local coil, are received by the RF system 920. The responsive magnetic resonance signals may be amplified, demodulated, filtered, and digitized under direction of commands produced by the pulse sequence server 910. The RF system 920 includes an RF transmitter for producing a wide variety of RF pulses used in MRI pulse sequences. The RF transmitter is responsive to the prescribed scan and direction from the pulse sequence server 910 to produce RF pulses of the desired frequency, phase, and pulse amplitude waveform. The generated RF pulses may be applied to the whole-body RF coil 928 or to one or more local coils or coil arrays.

The RF system 920 also includes one or more RF receiver channels. An RF receiver channel includes an RF preamplifier that amplifies the magnetic resonance signal received by the coil 928 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{( {I}^{2} + {Q}^{2} )}$$

and the phase of the received magnetic resonance signal may also be determined according to the following relationship:

$$ϕ = {tan}^{- 1} ( \frac{Q}{I} )$$

The pulse sequence server 910 may receive patient data from a physiological acquisition controller 930. By way of example, the physiological acquisition controller 930 may receive signals from a number of different sensors connected to the patient, including electrocardiograph (“ECG”) signals from electrodes, or respiratory signals from a respiratory bellows or other respiratory monitoring devices. These signals may be used by the pulse sequence server 910 to synchronize, or “gate,” the performance of the scan with the subject's heartbeat or respiration.

The pulse sequence server 910 may also connect to a scan room interface circuit 932 that receives signals from various sensors associated with the condition of the patient and the magnet system. Through the scan room interface circuit 932, a patient positioning system 934 can receive commands to move the patient to desired positions during the scan.

The digitized magnetic resonance signal samples produced by the RF system 920 are received by the data acquisition server 912. The data acquisition server 912 operates in response to instructions downloaded from the operator workstation 902 to receive the real-time magnetic resonance data and provide buffer storage, so that data are not lost by data overrun. In some scans, the data acquisition server 912 passes the acquired magnetic resonance data to the data processor server 914. In scans that require information derived from acquired magnetic resonance data to control the further performance of the scan, the data acquisition server 912 may be programmed to produce such information and convey it to the pulse sequence server 910. For example, during pre-scans, magnetic resonance data may be acquired and used to calibrate the pulse sequence performed by the pulse sequence server 910. As another example, navigator signals may be acquired and used to adjust the operating parameters of the RF system 920 or the gradient system 918, or to control the view order in which k-space is sampled. In still another example, the data acquisition server 912 may also process magnetic resonance signals used to detect the arrival of a contrast agent in a magnetic resonance angiography (“MRA”) scan. For example, the data acquisition server 912 may acquire magnetic resonance data and processes it in real-time to produce information that is used to control the scan.

The data processing server 914 receives magnetic resonance data from the data acquisition server 912 and processes the magnetic resonance data in accordance with instructions provided by the operator workstation 902. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 914 are conveyed back to the operator workstation 902 for storage. Real-time images may be stored in a data base memory cache, from which they may be output to operator display 902 or a display 936. Batch mode images or selected real time images may be stored in a host database on disc storage 938. When such images have been reconstructed and transferred to storage, the data processing server 914 may notify the data store server 916 on the operator workstation 902. The operator workstation 902 may be used by an operator to archive the images, produce films, or send the images via a network to other facilities.

The MRI system 900 may also include one or more networked workstations 942. For example, a networked workstation 942 may include a display 944, one or more input devices 946 (e.g., a keyboard, a mouse), and a processor 948. The networked workstation 942 may be located within the same facility as the operator workstation 902, or in a different facility, such as a different healthcare institution or clinic.

The networked workstation 942 may gain remote access to the data processing server 914 or data store server 916 via the communication system 940. Accordingly, multiple networked workstations 942 may have access to the data processing server 914 and the data store server 916. In this manner, magnetic resonance data, reconstructed images, or other data may be exchanged between the data processing server 914 or the data store server 916 and the networked workstations 942, such that the data or images may be remotely processed by a networked workstation 942.

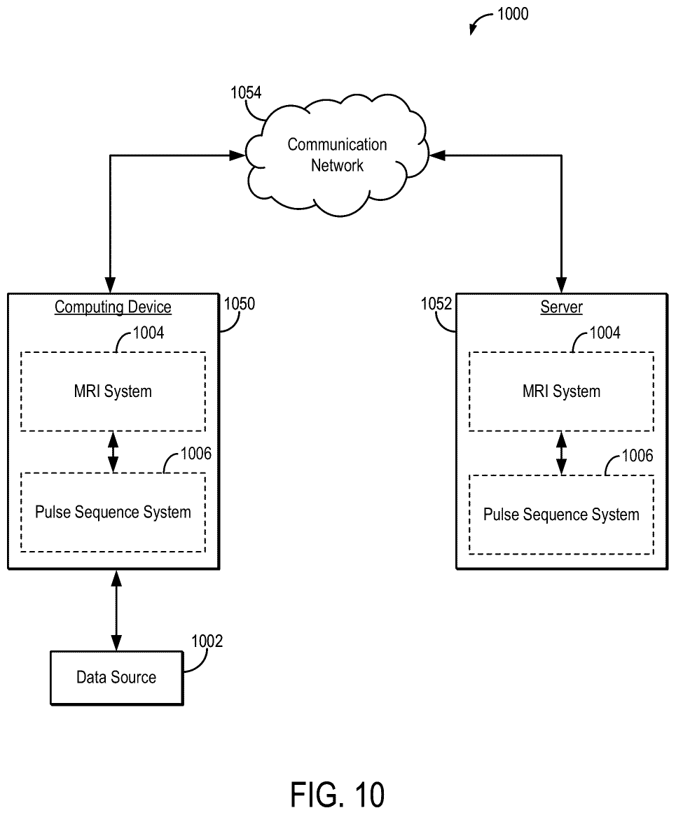

Referring now to FIG. 10, an example of an MRI system 1000 is shown, which may be used in accordance with some aspects of the systems and methods described in the present disclosure. As shown in FIG. 10, a computing device 1050 can receive one or more types of data (e.g., pulse sequence parameters, relaxation parameters, off-resonance or B₀ inhomogeneity data, signal evolution data, k-space data, receiver coil sensitivity data) from data source 1002.

In some configurations, computing device 1050 can execute at least a portion of a pulse sequence design system 1006 to determine pulse sequence parameters (e.g., including RF pulse shapes) that can be output or used by the MRI system 1004 to acquire imaging data. In some configurations, computing device 1050 can execute at least a portion of an MRI system 1004 to reconstruct images from magnetic resonance data (e.g., k-space data) acquired using jointly optimized pulses. In some configurations, the MRI system 1004 can implement an automated pipeline to provide MRI images, MRF maps, MRF synthetic images, etc.

Additionally or alternatively, in some configurations, the computing device 1050 can communicate information about data received from the data source 1002 to a server 1052 over a communication network 1054, which can execute at least a portion of the pulse sequence design system 1006 or MRI system 1004. In such configurations, the server 1052 can return information to the computing device 1050 (and/or any other suitable computing device) indicative of an output of the pulse sequence design system 1006 or MRI system 1004.

In some configurations, computing device 1050 and/or server 1052 can be any suitable computing device or combination of devices, such as a desktop computer, a laptop computer, a smartphone, a tablet computer, a wearable computer, a server computer, a virtual machine being executed by a physical computing device, and so on. The computing device 1050 and/or server 1052 can also reconstruct images from the data.

In some configurations, data source 1002 can be any suitable source of data (e.g., measurement data, images reconstructed from measurement data, processed image data), such as an MRI system, another computing device (e.g., a server storing measurement data, images reconstructed from measurement data, processed image data), and so on. In some configurations, data source 1002 can be local to computing device 1050. For example, data source 1002 can be incorporated with computing device 1050 (e.g., computing device 1050 can be configured as part of a device for measuring, recording, estimating, acquiring, or otherwise collecting or storing data). As another example, data source 1002 can be connected to computing device 1050 by a cable, a direct wireless link, and so on. Additionally or alternatively, in some configurations, data source 1002 can be located locally and/or remotely from computing device 1050, and can communicate data to computing device 1050 (and/or server 1052) via a communication network (e.g., communication network 1054).

In some configurations, communication network 1054 can be any suitable communication network or combination of communication networks. For example, communication network 1054 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 1054 can be a local area network, a wide area network, a public network (e.g., the Internet), a private or semi-private network (e.g., a corporate or university intranet), any other suitable type of network, or any suitable combination of networks. Communications links shown in FIG. 10 can each be any suitable communications link or combination of communications links, such as wired links, fiber optic links, Wi-Fi links, Bluetooth links, cellular links, and so on.

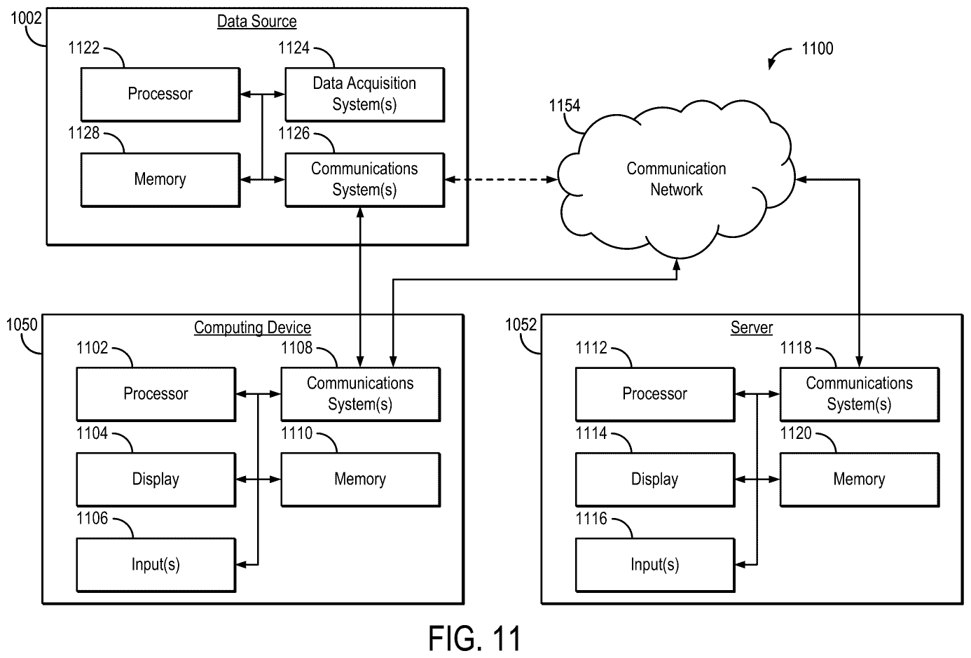

Referring now to FIG. 11, an example of hardware 1100 that can be used to implement data source 1002, computing device 1050, and server 1052 in accordance with some configurations of the systems and methods described in the present disclosure is shown.

As shown in FIG. 11, in some configurations, computing device 1050 can include a processor 1102, a display 1104, one or more inputs 1106, one or more communication systems 1108, and/or memory 1110. In some configurations, processor 1102 can be any suitable hardware processor or combination of processors, such as a central processing unit (“CPU”), a graphics processing unit (“GPU”), and so on. In some configurations, display 1104 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 1106 can include any suitable input devices and/or sensors that can be used to receive user input, such as a keyboard, a mouse, a touchscreen, a microphone, and so on.

In some configurations, communications systems 1108 can include any suitable hardware, firmware, and/or software for communicating information over communication network 1054 and/or any other suitable communication networks. For example, communications systems 1108 can include one or more transceivers, one or more communication chips and/or chip sets, and so on. In a more particular example, communications systems 1108 can include hardware, firmware, and/or software that can be used to establish a Wi-Fi connection, a Bluetooth connection, a cellular connection, an Ethernet connection, and so on.

In some configurations, memory 1110 can include any suitable storage device or devices that can be used to store instructions, values, data, or the like, that can be used, for example, by processor 1102 to present content using display 1104, to communicate with server 1052 via communications system(s) 1108, and so on. Memory 1110 can include any suitable volatile memory, non-volatile memory, storage, or any suitable combination thereof. For example, memory 1110 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 1110 can have encoded thereon, or otherwise stored therein, a computer program for controlling operation of computing device 1050. In such configurations, processor 1102 can execute at least a portion of the computer program to present content (e.g., images, user interfaces, graphics, tables), receive content from server 1052, transmit information to server 1052, and so on. For example, the processor 1102 and the memory 1110 can be configured to perform the methods described herein.

In some configurations, server 1052 can include a processor 1112, a display 1114, one or more inputs 1116, one or more communications systems 1118, and/or memory 1120. In some configurations, processor 1112 can be any suitable hardware processor or combination of processors, such as a CPU, a GPU, and so on. In some configurations, display 1114 can include any suitable display devices, such as an LCD screen, LED display, OLED display, electrophoretic display, a computer monitor, a touchscreen, a television, and so on. In some configurations, inputs 1116 can include any suitable input devices and/or sensors that can be used to receive user input, such as a keyboard, a mouse, a touchscreen, a microphone, and so on.

In some configurations, communications systems 1118 can include any suitable hardware, firmware, and/or software for communicating information over communication network 1054 and/or any other suitable communication networks. For example, communications systems 1118 can include one or more transceivers, one or more communication chips and/or chip sets, and so on. In a more particular example, communications systems 1118 can include hardware, firmware, and/or software that can be used to establish a Wi-Fi connection, a Bluetooth connection, a cellular connection, an Ethernet connection, and so on.

In some configurations, memory 1120 can include any suitable storage device or devices that can be used to store instructions, values, data, or the like, that can be used, for example, by processor 1112 to present content using display 1114, to communicate with one or more computing devices 1050, and so on. Memory 1120 can include any suitable volatile memory, non-volatile memory, storage, or any suitable combination thereof. For example, memory 1120 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 1120 can have encoded thereon a server program for controlling operation of server 1052. In such configurations, processor 1112 can execute at least a portion of the server program to transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone), and so on.

In some configurations, the server 1052 is configured to perform the methods described in the present disclosure. For example, the processor 1112 and memory 1120 can be configured to perform the methods described herein.

In some configurations, data source 1002 can include a processor 1122, one or more data acquisition systems 1124, one or more communications systems 1126, and/or memory 1128. In some configurations, processor 1122 can be any suitable hardware processor or combination of processors, such as a CPU, a GPU, and so on. In some configurations, the one or more data acquisition systems 1124 are generally configured to acquire data, images, or both, and can include an MRI system. Additionally or alternatively, in some configurations, the one or more data acquisition systems 1124 can include any suitable hardware, firmware, and/or software for coupling to and/or controlling operations of an MRI system. In some configurations, one or more portions of the data acquisition system(s) 1124 can be removable and/or replaceable.

Note that, although not shown, data source 1002 can include any suitable inputs and/or outputs. For example, data source 1002 can include input devices and/or sensors that can be used to receive user input, such as a keyboard, a mouse, a touchscreen, a microphone, a trackpad, a trackball, and so on. As another example, data source 1002 can include any suitable display devices, such as an LCD screen, an LED display, an OLED display, an electrophoretic display, a computer monitor, a touchscreen, a television, etc., one or more speakers, and so on.

In some configurations, communications systems 1126 can include any suitable hardware, firmware, and/or software for communicating information to computing device 1050 (and, in some configurations, over communication network 1054 and/or any other suitable communication networks). For example, communications systems 1126 can include one or more transceivers, one or more communication chips and/or chip sets, and so on. In a more particular example, communications systems 1126 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 1128 can include any suitable storage device or devices that can be used to store instructions, values, data, or the like, that can be used, for example, by processor 1122 to control the one or more data acquisition systems 1124, and/or receive data from the one or more data acquisition systems 1124; to generate images from data; present content (e.g., data, images, a user interface) using a display; communicate with one or more computing devices 1050; and so on. Memory 1128 can include any suitable volatile memory, non-volatile memory, storage, or any suitable combination thereof. For example, memory 1128 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 1128 can have encoded thereon, or otherwise stored therein, a program for controlling operation of medical image data source 1002. In such configurations, processor 1122 can execute at least a portion of the program to generate images, transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and Care elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method for using a processor to prepare a magnetic resonance imaging (MRI) pulse sequence for performing imaging of a patient, the method including steps comprising:
defining a target slice profile across a slice dimension of an imaging target;
simulating a pulse sequence comprising a plurality of radiofrequency (RF) pulses, wherein simulating the pulse sequence comprises calculating a plurality of slice profiles produced in response to the plurality of RF pulses;
determining pulse parameters of each of the plurality of RF pulses by reducing a cost function including a comparison term that calculates differences between the plurality of slice profiles and a target term that calculates a difference between the target slice profile and at least one of the plurality of slice profiles.

2. The method of claim 1, further comprising performing the pulse sequence using the pulse parameters of each of the plurality of RF pulses to acquire MRI data from the imaging target.

3. The method of claim 1, wherein calculating each of the plurality of slice profiles accounts for each of the plurality of slice profiles preceding in time within the pulse sequence.

4. The method of claim 1, wherein the RF pulses are refocusing pulses.

5. The method of claim 1, wherein the comparison term measures a rank of a combination of transverse magnetization of the plurality of slice profiles.

6. The method of claim 1, wherein the target term weights the difference between the target slice profile and the at least one of the plurality of slice profiles according to at least one of a magnetization relaxation of the imaging target or a flip angle of the at least one of the plurality of slice profiles.

7. The method of claim 1, further comprising scaling the plurality of slice profiles to have similar amplitudes prior to determining the pulse parameters.

8. The method of claim 1, wherein simulating the pulse sequence comprises generating a forward model that relates the plurality of RF pulses with the plurality of slice profiles using differentiable tensor operations.

9. The method of claim 1, wherein simulating the pulse sequence comprises performing at least one of a Bloch simulation, an extended phase graph (EPG) algorithm, or a spinor-EPG algorithm.

10. A method for using a processor to prepare a magnetic resonance imaging (MRI) pulse sequence for performing imaging of a patient, the method including steps comprising:
defining a target slice profile across a slice dimension of an imaging target;
simulating a first echo having a first slice profile generated at a first echo time in response to a first one or more radiofrequency (RF) pulses
simulating a second echo having a second slice profile generated at a second echo time in response to a second one or more RF pulses; and
determining pulse parameters of at least one of the first one or more RF pulses or the second one or more RF pulses for acquiring MRI data from the imaging target by reducing a cost function including a comparison term that calculates a difference between the first slice profile and the second slice profile as a function of the slice dimension and a target term that calculates a difference between the target slice profile and at least one of the first slice profile and the second slice profile as a function of the slice dimension.

11. The method of claim 10, wherein the first one or more RF pulses and the second one or more RF pulses form an echo train of a pulse sequence in which the second echo occurs after the first echo.

12. The method of claim 10, wherein the first one or more RF pulses are part of a first pulse sequence, and the second one or more RF pulses are part of a second pulse sequence different than the first pulse sequence.

13. The method of claim 12, wherein the first pulse sequence is a gradient echo sequence, and the first one or more RF pulses comprise an excitation pulse, and wherein the second pulse sequence is a spin echo sequence, and the second one or more RF pulses comprise an excitation pulse and a refocusing pulse.

14. The method of claim 10, wherein simulating the first slice profile comprises generating a forward model that relates the first one or more RF pulses with the first slice profile and the second one or more RF pulses with the second slice profile using differentiable tensor operations.

15. The method of claim 10, wherein the comparison term measures a rank of a combination of transverse magnetization of at least the first slice profile and the second slice profile.

16. The method of claim 10, wherein the target term weights the difference between the target slice profile and the at least one of the first slice profile and the second slice profile.

17. The method of claim 10, wherein the target slice profile comprises a magnitude and a phase.

18. The method of claim 10, wherein simulating the first echo comprises performing at least one of a Bloch simulation, an extended phase graph (EPG) algorithm, or a spinor-EPG algorithm and simulating the second echo comprises performing at least one of a Bloch simulation, an EPG algorithm, or a spinor-EPG algorithm.

19. The method of claim 10, further comprising performing the first one or more RF pulses and the second one or more RF pulses to acquire the MRI data from the imaging target.

20. A computer-readable storage medium having stored thereon a computer program that, when executed by a computer processor, causes the processor to carry out steps comprising:
receiving a target slice profile across a slice dimension of an imaging target;
simulating a first echo having a first slice profile generated at a first echo time in response to a first one or more radiofrequency (RF) pulses;
simulating a second echo having a second slice profile generated at a second echo time in response to a second one or more RF pulses; and
determining pulse parameters of at least one of the first one or more RF pulses or the second one or more RF pulses for acquiring MRI data from the imaging target by reducing a cost function including a comparison term that calculates a difference between the first slice profile and the second slice profile as a function of the slice dimension and a target term that calculates a difference between the target slice profile and at least one of the first slice profile and the second slice profile as a function of the slice dimension.

21. The computer-readable storage medium of claim 20, wherein simulating the second echo further accounts for the first one or more RF pulses.

22. The computer-readable storage medium of claim 20, wherein simulating the first slice profile comprises generating a forward model that relates the first one or more RF pulses with the first slice profile and the second one or more RF pulses with the second slice profile using differentiable tensor operations.

23. The computer-readable storage medium of claim 20, wherein the comparison term measures a rank of a combination of transverse magnetization of the first slice profile and the second slice profile.

24. The computer-readable storage medium of claim 20, wherein simulating the first echo comprises performing at least one of a Bloch simulation, an extended phase graph (EPG) algorithm, or a spinor-EPG algorithm and simulating the second echo comprises performing at least one of a Bloch simulation, an EPG algorithm, or a spinor-EPG algorithm.
